(19) United States
(12) Patent Application Publication
(10) Pub. No.: US 2005/0065576 A1
(43) Pub. Date: Mar. 24, 2005

(54) Counterbalancing device for tanning systems

(76) Inventor: Alain Lamoureux, St-Eustache (CA)

Correspondence Address: Alain Lamoureux, 440, Guindon # 106, St-Eustache, QC, J7R 6S5, CA

(21) Appl. No.: 10/665,621

(22) Filed: Sep. 22, 2003

(51) Int. Cl. A61N 1/00

(52) U.S. Cl. 607/88

(57) ABSTRACT

A counterbalancing device for use in conjunction with a tanning system having a stationary lower unit and a pivotable upper unit pivotally attached to the lower unit by a unit hinge. The device includes a first component and a second component. A device hinge hingedly connects the first and second components together for relative pivotal movement therebetween. A biasing structure generates a biasing force urging the first and second components towards the device open configuration. A component-to-unit coupling structure operatively couples the first and second components respectively to the upper and lower units. The biasing structure is calibrated so that the biasing force substantially counterbalances the weight of the upper unit.

### FIELD OF THE INVENTION

The present invention relates to the general field of ultraviolet light tanning systems and is particularly concerned with a counterbalancing device for tanning systems.

### BACKGROUND OF THE INVENTION

Tanning systems making use of artificial light in one form or another have been in vogue for an appreciable length of time. Such tanning systems are particularly popular in countries wherein the summer tanning season is relatively short especially during winter months when a natural tan cannot be readily acquired.

Most conventional tanning systems include a stationary lower tanning unit mounted on a base and a movable upper tanning unit. The upper tanning unit has a row of lamps that are intended to tan the upwardly facing part of the user while the lower tanning unit has rows of lamps that are intended to tan the downwardly facing part of the user. The upper tanning unit is typically connected by hinges to the lower tanning unit so that the tanning system can be opened and closed in a manner somewhat analogous to a clam shell.

When the tanning system is in the open position, a user can enter between the upper and lower tanning units to lie down on the lower tanning unit. The upper tanning unit is then moved towards the lower tanning unit until it reaches a closed position wherein the upper and lower tanning units form an internal tanning chamber where the user is partially surrounded by tanning lamps.

Although somewhat satisfactory, most conventional tanning systems suffer from important drawbacks. One of such drawbacks relates to the difficulty for many users to raise and lower the somewhat heavy upper tanning unit. Indeed, especially when the upper tanning unit is massive, the force required to lift the upper tanning unit or to gently lower the latter may be considerable.

Also, some users, because of claustrophobia or other reasons prefer to maintain the upper tanning unit in a somewhat spaced relationship relative to the lower tanning unit instead of the fully closed or lowered position. Hence, in certain situations, it may be desirable to retain the upper tanning unit in any instantaneous position between the fully raised and fully lowered position.

The need for facilitating the raising and lowering of the upper tanning unit as well as allowing for retention of the upper tanning unit in an intermediate position has been recognised in the prior art. Accordingly, some prior art tanning systems are provided with counterbalancing means.

For example, some prior art tanning systems are provided with a motor for mechanically raising and lowering the upper tanning unit. The use of such motors however unduly increases the overall manufacturing and maintenance costs of the tanning system. In addition, in the event of a motor failure or a power outage, there exists the possibility that the user may be trapped in the tanning system with the upper tanning unit in its lowered position.

Other tanning systems use a counterbalancing lifting piston in order to facilitate the raising of the upper tanning unit. The use of lifting pistons or cylinders also increases the overall manufacturing costs. Moreover, most piston-type counterbalancing systems employ gas cylinders which are relatively sensitive to the ambient temperature. The tanning system radiating relatively large amount heat may cause the ambient temperature to fluctuate significantly. Hence, the efficiency of the piston-type cylinders in assisting the lifting of the upper tanning unit may very significantly and the ability of such piston-type systems to maintain the tanning unit in an intermediate desired position may also vary significantly. In addition, listing pistons are prone to fail after a relatively short period of use and, therefore, must be replaced on a somewhat regular basis, increasing maintenance costs.

Prior art counterbalancing means using a motor or a piston-type mechanism are also typically inherently obstructive and may interfere with free access to the tanning chamber formed by the upper and lower tanning units. Furthermore, such motor or piston-type counterbalancing mechanisms often deter the overall aesthetical appearance of the tanning system.

Problems associated with motor and piston-type counterbalancing mechanisms has also been recognised in the prior art. Accordingly, some tanning systems have resorted to using counterbalancing springs operatively coupled to the upper tanning unit to provide a lever-type counterbalance.

Most prior art spring-type counterbalancing mechanisms however suffer from numerous drawbacks including overall complexity, being relatively unreliable, being potentially dangerous in the event of a component failure, being substantially obstructive and deterring the overall aesthetical appearance of the tanning system. Most prior art spring-type counterbalancing mechanisms also suffer from lack of adjustability and lack of potential for customisation depending on the weight of the upper tanning unit and/or the preferences of the intended user. Accordingly, there exists a need for a tanning system having an improved counterbalancing mechanism.

Another drawback commonly associated with most prior art tanning systems resides in that replacement of the tanning lamps is typically a lengthy and tedious operation. Accordingly, there exists a need for a tanning system allowing for facilitated replacement of the tanning lamps.

Another drawback associated with some conventional tanning systems relates to the inherent lack of versatility in terms of customisation of the light ray pattern directed towards the intended user. Indeed, in some situations, it may be desirable to provide increased tanning rays to specific regions of the user body. For example, some individuals may require a darker tan in the face region.

In the past, it has been necessary for an intended user wanting a darker tan in a specific region to utilise a separate tanning unit such as a separate facial tanning unit before or after utilising the conventional tanning bed or system. Accordingly, there exists a need for a tanning system allowing the possibility of providing tanning rays of various intensities directed towards selected body parts.

### SUMMARY OF THE INVENTION

In accordance with the present invention, there is provided a counterbalancing device for use in conjunction with a tanning system, the tanning system generating tanning light rays for tanning an intended user, the tanning system having a stationary lower unit and a pivotable upper unit pivotally attached to the lower unit by a unit hinge for pivotable movement therebetween about a unit pivot axis, the upper and lower units respectively defining longitudinally extending upper and lower unit front and rear ends, the upper and lower units respectively defining transversally extending upper and lower unit opposed side ends, the counterbalancing device comprising: a first component, the first component having a first component base wall, the first component base wall defining a first component inner surface and a substantially opposed first component outer surface; a second component, the second component having a second component base wall, the second component base wall defining a second component inner surface and a substantially opposed second component outer surface; a device hinge means for hingedly connecting the first and second components together for relative pivotal movement therebetween about a component pivot axis between a device closed configuration wherein the first and second component inner surfaces are in a substantially parallel and proximal relationship relative to each other and a device open configuration wherein the first and second component inner surfaces are in a substantially angled and spaced relationship relative to each other; a biasing means for generating a biasing force urging the first and second components towards the device open configuration, the biasing means having a biasing means first abutment segment, a substantially opposed biasing means second abutment segment and a biasing means biasing segment extending integrally therebetween; a spring-to-component coupling means for operatively coupling the spring first and second abutment segments in abutting contact respectively with the first and second component inner surfaces; a component-to-unit coupling means for operatively coupling the first and second components respectively to the upper and lower units; whereby the biasing means is calibrated so that the biasing force substantially counterbalances the weight of the upper unit.

Conveniently, the biasing means includes a torsion spring, the torsion spring having a spring first abutment segment, a substantially opposed spring second abutment segment and a spring coiled segment extending integrally therebetween, the spring first and second abutment segments being configured, sized and positioned for abuttingly contacting respectively the first and second component inner surfaces, the spring coiled segment being able to generate a biasing force for biasing the first and second components towards the device open configuration.

Typically, the biasing means includes at least two independent torsion springs operatively coupled to the first and second components so as to independently work in parallel to commonly generate the biasing force.

Conveniently, the spring-to-component coupling means includes a first “U”-shaped bolt and a second “U”-shaped bolt each having a pair of bolt legs; a pair of first spring attachment apertures extending through the first component base wall, the first spring attachment apertures being configured, sized and positioned for receiving the pair of bolt legs of the first “U”-shaped bolt; a pair of second spring attachment apertures extending through the second component base wall, the second spring attachment apertures being configured, sized and positioned for receiving the pair of bolt legs of the second “U”-shaped bolt.

Typically, the first component base wall defines a pair of longitudinally opposed first base wall end edges and the second component base wall defines a pair of longitudinally opposed second base wall end edges; the first component base wall being provided with a first base wall end flange extending substantially perpendicularly from each of the first base wall end edges; the second component base wall being provided with a second base wall end flange extending substantially perpendicularly from each of the second base wall end edges; each of the first base wall end flanges being provided with a first end flange hinge segment projecting substantially outwardly therefrom; each of the second base wall end flanges being provided with a second end flange hinge segment projecting substantially outwardly therefrom; each pair of adjacent first and second flange hinge segments being substantially in register with each other in a substantially proximal and parallel relationship relative to each other; the device hinge means including a device hinge axle pivotally connecting together each pair of adjacent first and second flange hinge segments.

Conveniently, the unit hinge includes an upper unit hinge arm extending from the upper unit rear end substantially adjacent each of the upper unit side ends; a lower unit hinge arm extending from the lower unit rear end substantially adjacent each of the lower unit side ends; a unit hinge axle pivotally connecting together each pair of adjacent upper and lower unit hinge arms.

Typically, the first component base wall defines a pair of longitudinally opposed first base wall end edges and the second component base wall defines a pair of longitudinally opposed second base wall end edges; the first component base wall being provided with a first base wall end flange extending substantially perpendicularly from each of the first base wall end edges; the second component base wall being provided with a second base wall end flange extending substantially perpendicularly from each of the first base wall end edges; each of the first base wall end flange being provided with a first end flange hinge segment projecting substantially outwardly therefrom; each of the second base wall end flange being provided with a second end flange hinge segment projecting substantially outwardly therefrom; each pair of adjacent first and second flange hinge segments being substantially in register with each other in a substantially proximal and parallel relationship relative to each other; the device hinge means including a device hinge axle pivotally connecting together each pair of adjacent first and second flange hinge segments; the device and unit hinge axles being in a substantially collinear relationship relative to each other.

Conveniently, the first and second end flange hinge segments are respectively provided with first and second end flange hinge apertures extending therethrough; the upper and lower unit hinge arms are respectively provided with upper and lower unit hinge apertures extending therethrough;—a pair of adjacent first and second end flange hinge apertures is positioned substantially in register with a corresponding pair of upper and lower unit hinge apertures so that an hinge bolt acts as both the device and unit hinge axles.

Typically, the device further comprises an upper unit hinge arm extending from the upper unit rear end substantially adjacent each of the upper unit side ends; each of the upper unit hinge arm including an upper arm first segment attached to the upper unit, an upper arm second segment extending substantially downwardly at an angle relative to the upper arm first segment from a positioned located substantially adjacent the upper unit rear end, an upper arm third segment extending substantially rearwardly from the upper arm second segment; a lower unit hinge arm extending from the lower unit rear end substantially adjacent each of the lower unit side ends; each of the lower unit hinge arm including an lower arm first segment attached to the lower unit, an lower arm second segment extending substantially upwardly at an angle relative to the lower arm first segment from a positioned located substantially adjacent the lower unit rear end, an lower arm third segment extending substantially rearwardly from the lower arm second segment; a unit hinge axle pivotally connecting together the upper and lower arm second segments so that the upper and lower arm first and third segments pivot together in a scissor-like manner.

Conveniently, the device further comprises a third segment-to-component coupling means extending between the upper and lower arm third segments and the second and first components for coupling the upper and lower arm third segments respectively to the second and first components.

Typically, the first component base wall defines a pair of longitudinally opposed first base wall end edges and the second component base wall defines a pair of longitudinally opposed second base wall end edges; the first component base wall being provided with a first base wall end flange extending substantially perpendicularly from each of the first base wall end edges; the second component base wall being provided with a second base wall end flange extending substantially perpendicularly from each of the first base wall end edges; each of the first base wall end flange being provided with a first end flange hinge segment projecting substantially outwardly therefrom; each of the second base wall end flange being provided with a second end flange hinge segment projecting substantially outwardly therefrom; each pair of adjacent first and second flange hinge segments being substantially in register with each other in a substantially proximal and parallel relationship relative to each other; the device hinge means including a device hinge axle pivotally connecting together each pair of adjacent first and second flange hinge segments; the device and unit hinge axles being in a substantially collinear relationship relative to each other; the counterbalancing device also including a third segment-to-component coupling means extending between one of the upper and lower arm third segments and the second and first components for coupling the one of the upper and lower arm third segments respectively to the second and first components.

Conveniently, the third segment-to-component coupling means includes an upper arm coupling aperture and a lower arm coupling aperture extending respectively through the one of the upper and lower arm third segments; a first end flange coupling aperture and a second end flange coupling aperture extending respectively through the first and second end flanges; a first arm-to-flange coupling bolt and a second arm-to-flange coupling bolt extending respectively though the upper arm coupling aperture and the second end flange coupling aperture and through the lower arm coupling aperture and the first end flange coupling aperture; a first end flange hinge aperture and a second end flange hinge aperture extending respectively through the first and second end flanges; an upper unit hinge aperture and a lower unit hinge aperture extending respectively through the upper and lower unit hinge arms; the pair of adjacent first and second hinge apertures is positioned substantially in register with a corresponding pair of upper and lower unit hinge apertures so that an hinge bolt acts as both the device and unit hinge axles.

Typically, the device further comprises a biasing force adjustment means for allowing adjustment of the magnitude of the biasing force. Typically, either one of the upper or lower arm coupling apertures has a substantially elongated and vertical configuration.

Conveniently, the lower unit includes at least one lower tanning lamp; a lower lamp chamber defining a lower chamber inner volume for housing the at least one lower tanning lamp, the lower lamp chamber having a lower chamber tanning aperture extending therethrough for allowing at least part of the tanning light rays emanating from the lower tanning lamp to pass therethrough; a substantially transparent lower supporting platform extending substantially across the lower chamber tanning aperture for supporting the intended user while allowing the at least part of the tanning light rays emanating from the lower tanning lamp to pass therethrough; the lower supporting platform being movably coupled to the lower lamp chamber for movement relative thereto between a lower platform open configuration allowing access to the lower chamber inner volume and a lower platform closed configuration preventing access to the lower chamber inner volume.

Typically, the lower supporting platform is pivotally attached to the lower lamp chamber for pivotal movement relative thereto about a lower platform pivotal axis

Conveniently, the device further comprises a lower platform supporting means coupled to the lower supporting platform for selectively maintaining the lower supporting platform in the lower platform open configuration.

Typically, the device further comprises a lower platform releasable locking means coupled to the lower supporting platform for releasably locking the lower supporting platform in the lower platform closed configuration.

Conveniently, the upper unit includes at least one upper tanning lamp; a upper lamp chamber defining a upper chamber inner volume for housing the at least one upper tanning lamp, the upper lamp chamber having a upper chamber tanning aperture extending therethrough for allowing at least part of the tanning light rays emanating from the upper tanning lamp to pass therethrough; a substantially transparent upper platform extending substantially across the upper chamber tanning aperture for allowing the at least part of the tanning light rays emanating from the upper tanning lamp to pass therethrough; the upper platform being movably coupled to the upper lamp chamber for movement relative thereto between a upper platform open configuration allowing access to the upper chamber inner volume and a upper platform closed configuration preventing access to the upper chamber inner volume.

Typically, the upper platform is pivotally attached to the upper lamp chamber for pivotal movement relative thereto about an upper platform pivotal axis

Conveniently, the devise further comprises a upper platform pivotal damping means coupled to the upper platform for damping the pivotal movement of the upper platform towards the upper platform open configuration.

Typically, the device further comprises a upper platform releasable locking means coupled to the upper platform for releasably locking the upper platform in the upper platform closed configuration.

In at least one embodiment of the invention, the devise comprises a high pressure lamp positioned within the upper lamp chamber substantially adjacent one of the upper unit side ends; a UV filter mounted to the upper platform, the UV filter being configured, positioned and sized so as to filter light rays emanating from the high pressure lamp when the upper platform is in the upper platform closed configuration.

Conveniently, the device further comprises a switch means for sensing the presence of the UV filter and only allowing the high pressure lamp to be turned on upon the detection of the presence of the UV filter.

Advantages of the present invention include that the proposed tanning system is provided with a counterbalancing mechanism facilitating the lowering and raising of the upper tanning unit relative to the lower tanning unit. Also, the proposed counterbalancing device allows for raising and lowering of the upper tanning unit through a set of quick, easy and ergonomical steps without requiring dexterity or excessive strength.

Furthermore, the proposed counterbalancing device allows the upper tanning unit to be retained in a plurality of instantaneous intermediate positions between the fully lowered and fully raised positions.

Also, the proposed counterbalancing system is specifically designed so as to be relatively safe even in the event of failure of one of its components such as failure of a spring component or the like.

The proposed counterbalancing device is designed so as to be adapted to also act as a hinge between the upper and lower tanning units. The proposed counterbalancing device is designed so as to be customisable to various contexts such as various upper tanning unit weights or user preferences. The proposed counterbalancing device is also designed so as to be relatively compact and non-obstructive.

The proposed counterbalancing device is also designed so as to be manufacturable through conventional forms of manufacturing so as to provide a counterbalancing device that is relatively simple, economical, long-lasting and relatively trouble-free in operation.

The proposed tanning system is also designed so as to facilitate replacement of the tanning lamps of the upper and/or lower tanning units through a set of quick and ergonomical steps without requiring special tooling or manual dexterity.

Still furthermore, the proposed tanning system, in at least one embodiment thereof, is intended to allow customisation of the tanning ray pattern so as to allow for selected user body parts to be subjected to tanning light of predetermined and variable intensity.

### BRIEF DESCRIPTION OF THE DRAWNGS

An embodiment of the present invention will now be disclosed, by way of example, in reference to the following drawings in which:

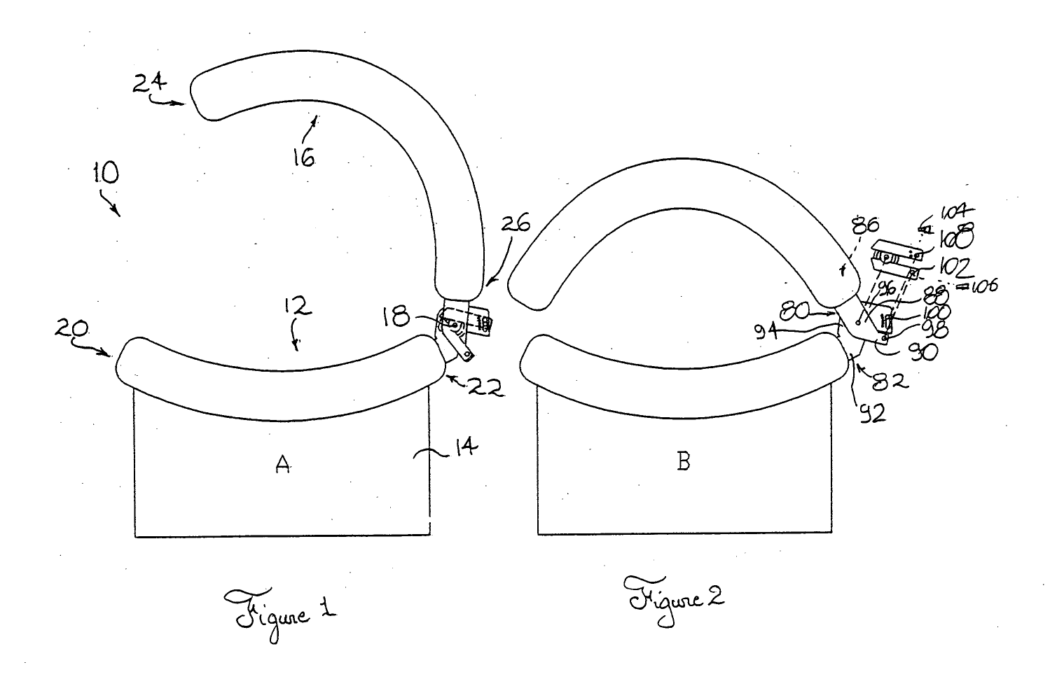

FIG. 1, in a side view, illustrates a tanning system in accordance with an embodiment of the present invention, the tanning system being shown with an upper unit thereof in a unit opened configuration;

FIG. 2, in a side view, illustrates the tanning system shown in FIG. 1 with its upper unit in a unit closed configuration;

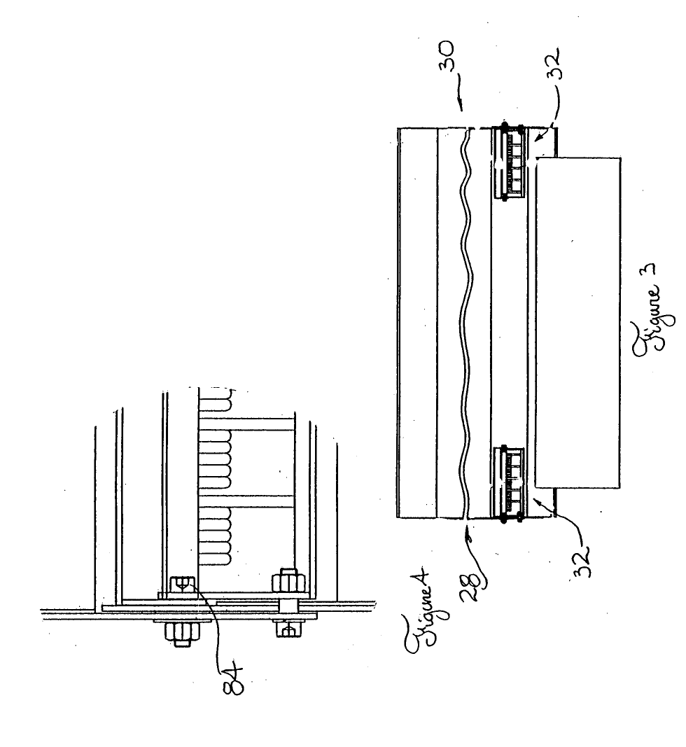

FIG. 3, in a rear view, illustrates the tanning system shown in FIGS. 1 and 2;

FIG. 4, in a detailed view with sections taken out, illustrates part of a counterbalancing device incorporated in a tanning system in accordance with an embodiment of the present invention;

FIG. 5, in a top view, illustrates a counterbalancing device in accordance with an embodiment of the present invention;

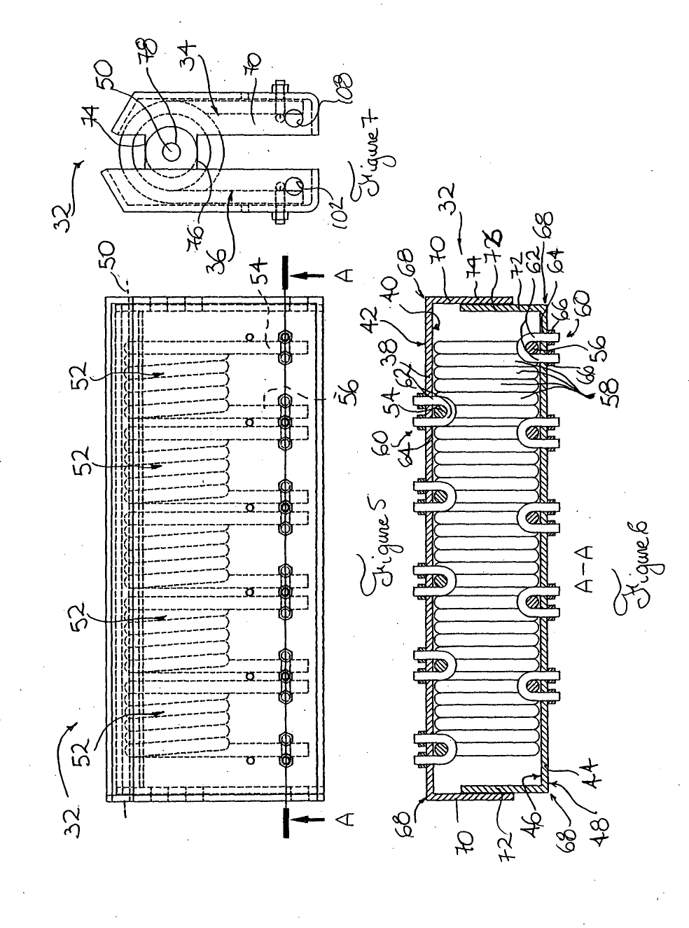

FIG. 6, in a longitudinal cross-sectional view taken along arrows A-A of FIG. 5, illustrates some of the features of the counterbalancing device shown in FIG. 5;

FIG. 7, in a side view, illustrates the counterbalancing device shown in FIGS. 5 and 6;

FIG. 8, in a front elevational view, illustrates a tanning system in accordance with an embodiment of the present invention, the tanning system being shown with its upper unit in an unit opened configuration and a lower supporting platform thereof also in an opened configuration;

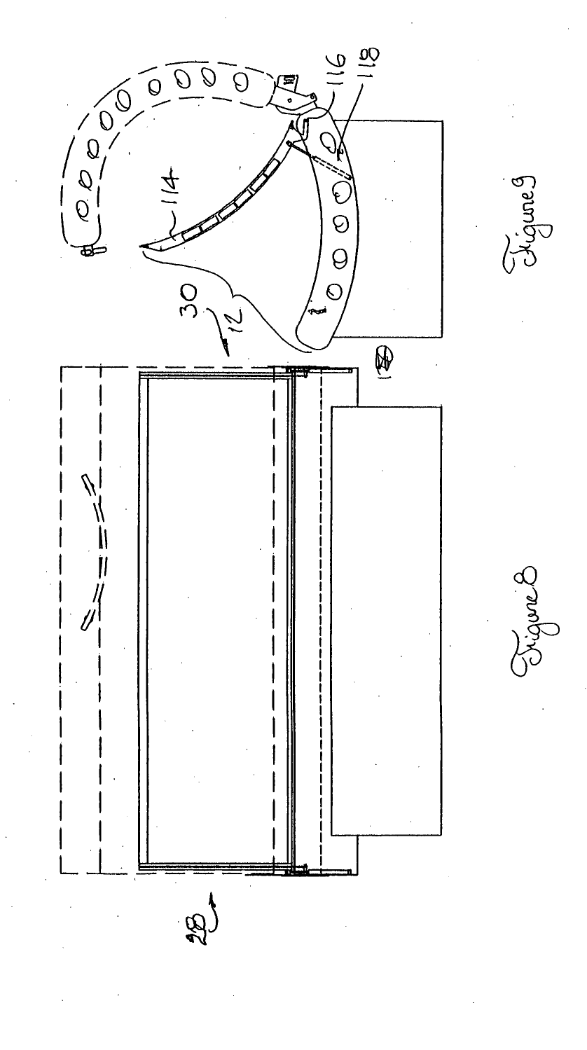

FIG. 9, in a side view, illustrates a tanning system in accordance with an embodiment of the present invention, the tanning system being shown with its upper unit in an unit opened configuration and a lower supporting platform thereof also in an opened configuration;

FIG. 10, in a front elevational view, illustrates a tanning system in accordance with an embodiment of the present invention, the tanning system being shown with its upper unit in an opened configuration and an upper platform thereof also in opened configuration;

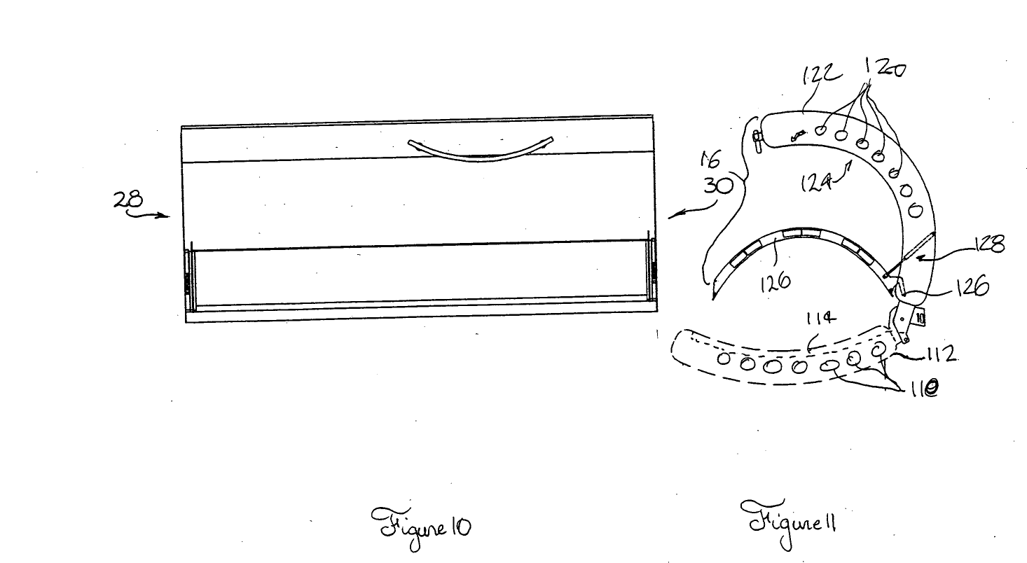

FIG. 11, in a side view, illustrates a tanning system in accordance with an embodiment of the present invention, the tanning system being shown with its upper unit in an opened configuration and an upper platform thereof also in opened configuration;

FIG. 12, in a side view, illustrates a tanning system in accordance with an embodiment of the present invention, the tanning system being shown with its upper platform in an unlocked configuration;

FIG. 13, in a partial front view, illustrates part of a tanning system in accordance with an embodiment of the present invention;

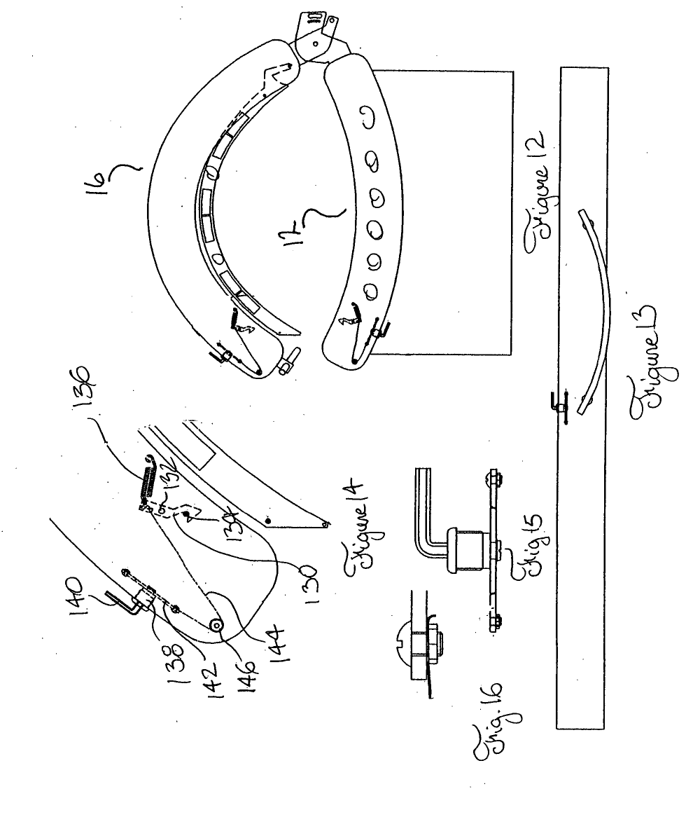

FIG. 14, in a detailed view with sections taken out, illustrates part of a platform locking mechanism incorporated in a tanning system in accordance with an embodiment of the present invention;

FIG. 15, in a detailed view, illustrates part of the platform locking mechanism shown in FIG. 14;

FIG. 16, in a detailed view, illustrates part of an attachment component incorporated in the platform locking mechanism shown in FIGS. 14 and 15;

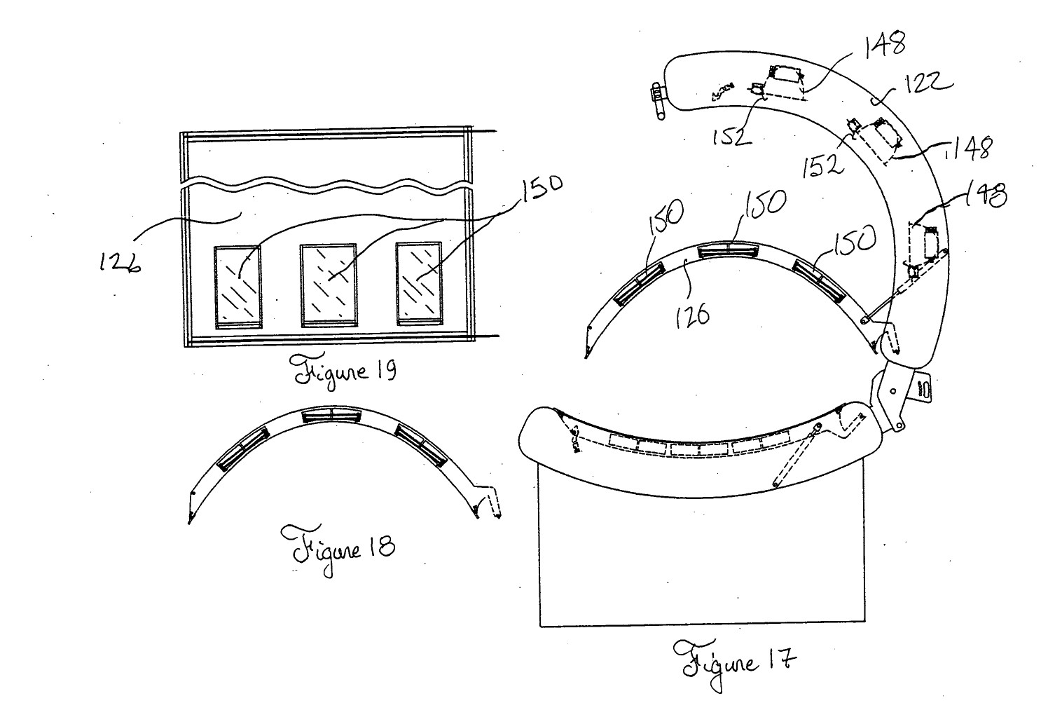

FIG. 17, in a side view, illustrates a tanning system in accordance with an alternative embodiment of the present invention, the tanning system being shown with its upper unit and upper platform both in an opened configuration;

FIG. 18 in a partial side view, illustrates an upper platform part of the tanning system shown in FIG. 17;

FIG. 19, in a partial top view with sections taken out, illustrates part of the upper platform shown in FIG. 18.

### DETAILED DESCRIPTION

Referring to FIG. 1, there is shown a tanning system in accordance with an embodiment of the present invention generally indicated by the reference numeral 10. The tanning system 10 is intended to generate tanning light rays for tanning an intended user (not shown). The tanning system 10 has a stationary lower unit 12 mounted on a base 14.

The tanning system 10 also has a pivotable upper unit 16 pivotally attached to the lower unit 12 by a unit hinge for pivotal movement therebetween about a unit pivot axis 18. The upper unit 16 is pivotable between a unit opened configuration shown in FIG. 1 allowing entry and exit of the user in the space defined between the upper and lower units 16, 12 and a unit closed configuration shown in FIG. 2 wherein the upper and lower units 16, 12 together form a tanning chamber.

The lower unit 12 defines longitudinally extending lower unit front and rear ends 20, 22. Similarly, the upper unit 16 defines longitudinally extending upper unit front and rear ends 24, 26. As illustrated more specifically in FIGS. 3, 8, and 10, the upper and lower units 16,12 respectively define transversally extending upper and lower unit opposite side ends 28, 30.

It should be understood that although the upper and lower units 16, 12 and the base 14 are shown as having specific configurations, the tanning system 10 could have other configurations without departing from the scope of the present invention.

As illustrated more specifically in FIG. 3, the tanning system 10 typically includes at least one and preferably two or more counterbalancing devices 32 for counterbalancing the weight of the upper unit 16. When a pair of counterbalancing devices 32 is used, each counterbalancing device 32 is typically positioned substantially adjacent the upper and lower unit opposite ends 28, 30.

As illustrated more specifically in FIGS. 5 through 7, each counterbalancing device 32 typically includes a first component 34 and a second component 36. The first component 34 has a first component base wall 38. The first component base wall 28 defines a first component inner surface 40 and a substantially opposed first components outer surface 42. Similarly, the second component 36 has a second component base wall 44. The second component base wall 44 defines a second component inner surface 46 and a substantially opposed second component outer surface 48.

The counterbalancing device 32 also includes a device hinge means for hingedly connecting the first and second components 34, 36 together for relative pivotal movement therebetween about a common pivot axis 50. The first and second components 34, 36 are pivotable in a jaw-like manner between a device opened configuration wherein the first and second component inner surfaces 40, 46 are in a substantially parallel and proximal relationship relative to each other and a device closed configuration wherein the first and second component inner surfaces 40, 46 are in a substantially angled and spaced relationship relative to each other.

The counterbalancing device 32 further includes biasing means for generating a biasing force urging the first and second components 34, 36 towards the device opened configuration. The biasing means has a biasing means first abutment segment, a substantially opposed biasing means second abutment segment and a biasing means biasing segment extending integrally therebetween.

The counterbalancing device 32 further includes a spring-to-component coupling means for operatively coupling the spring first and second abutment segments in bumping contact respectively with the first and second components inner surfaces 40, 46. The counterbalancing device 32 still further includes a component-to-unit coupling means for operatively coupling the first and second components 34, 36 respectively to the upper and lower units 16, 12.

The biasing means is typically calibrated so that the biasing force exerted thereby substantially counterbalances the weight of the upper unit 16. The counterbalancing device 10 hence facilitates movement of the upper unit 16 between the unit opened and closed configurations and also allows for the upper unit 16 to remain balanced in an intermediate position between the unit opened and closed configurations. The biasing means typically includes at least one and preferably a plurality of torsion springs 52. Each torsion spring has a spring first abutment segment 54, a substantially opposed spring second abutment segment 56 and a spring coiled segment 58 extending integrally therebetween. The spring first and second abutment segments 54, 56 are configured, sized and positioned for abuttingly contacting respectively the first and second component inner surfaces 40, 46. The spring coiled segment 58 is configured and sized for generating a biasing force biasing the first and second components 34, 36 towards the device opened configuration.

When a plurality of torsion springs 52 are used, the individual torsion springs 52 are operatively coupled to the first and second components 34, 36 so as to independently work in parallel to commonly generate the biasing force. This allows for customisation of the magnitude of the biasing force by varying the number of torsion springs used. Also, the use of more than one torsion spring 52 provides additional safety since, in the event of one of the torsion springs 52 becoming defective, the torsion springs 52 continue to exert a biasing force to the first and second components 34, 36.

Typically, the spring-to-component coupling means includes U-shaped bolts 60, each having a pair of bolt legs 62. The spring-to-component coupling means also includes first and second spring attachment apertures 64 extending respectively through the first and second component base walls 38, 44. The first and second spring attachment apertures 64 being configured, sized and positioned for receiving a corresponding pair of bolt legs 62. Nut components 66 are typically used for releasably securing the U-shaped bolts 60 respectively to the first and second component base walls 38, 44.

Each of the first and second component base walls 38, 44 typically defines a corresponding pair of longitudinally opposed first and second base wall end edges 68. The first component base wall is provided with a first base wall end flange 70 extending substantially perpendicularly from each of the first base wall end edges 68. Similarly, the second component base wall is typically provided with a second base wall end edge 72 substantially perpendicularly from each of the second base wall end edges 68.

As illustrated more specifically in FIG. 7, each of the first base wall end flanges 70 is provided with a corresponding first end flange hinge segment 74 projecting substantially outwardly therefrom. Similarly, each of the second base wall end flanges 72 is provided with a second end flange hinge segment 76 projecting substantially outwardly therefrom.

Each pair of adjacent first and second flange hinge segments 74, 76 is substantially in register with each other in substantially proximal and parallel relationship relative to each other. The device hinge means includes a device hinge axle 78 pivotally connecting together each pair of adjacent first and second flange hinge segments 74, 76. Typically, the device and unit hinge axles 18, 78 are in a substantially co-linear relationship relative to each other.

Typically, the unit hinge includes an upper unit hinge arm 80 extending from the upper unit rear end 26 substantially adjacent each of the upper unit side ends 28, 30. Similarly, the unit hinge also includes a lower unit hinge arm 82 extending from the lower unit rear end 22 substantially adjacent each of the lower unit side ends 28, 30. The unit hinge further includes a unit hinge axle typically connecting together each pair of adjacent upper and lower unit hinge arms 80, 82.

The first and second end flange hinge segments 74, 76 are respectively provided with first and second end flange hinge apertures extending therethrough. Similarly, the upper and lower unit hinge arms 80, 82 are respectively provided with upper and lower unit hinge apertures extending therethrough.

A pair of adjacent first and second end flange hinge apertures is positioned substantially in register with a corresponding pair of upper and lower unit hinge apertures so that a hinge bolt acts as both the device and unit hinge axles. The hinge bolt 84 is typically of the self-locking type so as to facilitate pivotal movement of the upper unit 16.

As illustrated more specifically in FIG. 2, each upper unit hinge arm 80 typically includes an upper arm first segment 86 attached to the upper unit 16, an upper arm second segment 88 extending substantially downwardly at an angle relative to the upper arm first segment 86 from a position located substantially adjacent the upper unit rear end 26. Each upper unit hinge arm 80 also includes an upper arm third segment 90 extending substantially rearwardly from the upper arm second segment 88.

Similarly, each lower unit hinge arm 82 includes a lower arm first segment 92 attached to the lower unit 12 and a lower arm second segment 94 extending substantially upwardly at an angle relative to the lower arm first segment 92 from a position located substantially adjacent the lower unit rear end 22. Each lower unit hinge arm 82 typically also includes a lower arm third segment 96 extending substantially rearwardly from the lower arm second segment 94. A unit hinge axle typically pivotally connects together the upper and lower arm second segments 88, 94 so that the upper and lower arm first and third segments 86, 92; 90, 96 pivot together in a scissor-like manner.

Each counterbalancing device 32 typically further includes a third segment-to-component coupling means extending between the upper and lower arm third segments 90, 96 and the second and first components 34, 32 for coupling the upper and lower arm third segments 90, 96 respectively to the second and first components 36, 34. The third segment-to-component coupling means typically includes an upper arm coupling aperture 98 and a lower arm coupling aperture 100 extending respectively through a corresponding one of the upper and lower arm third segments 90, 96. The third segment-to-component coupling means also includes a first end flange coupling aperture 102 and a second end flange coupling aperture 108 extending respectively through the first and second end flanges 70, 72.

The third segment-to-component coupling means further includes a first arm-to-flange coupling bolt 104 and a second arm-to-flange coupling bolt 106 extending respectively through the upper arm coupling aperture 98 and the second flange coupling aperture 102 and through the lower arm coupling aperture 100 and the first end flange coupling aperture 108.

Typically, the counterbalancing device 32 also includes a biasing force adjustment means for allowing adjustment of the magnitude of the biasing force. Typically, the biasing force adjustment means includes either one of the upper or lower arm coupling apertures 90, 100 having a substantially elongated and vertical configuration.

As illustrated more specifically in FIGS. 9, 11, and 12, the lower unit 12 includes at least one and preferably a set of lower tanning lamps 110 preferably positioned in a side-by-side relationship relative to each other. The lower unit 12 also includes a lower lamp chamber 112 defining a lower chamber inner volume for housing the lower tanning lamps 110. The lower lamp chamber 112 has a lower chamber aperture extending therethrough for allowing at least a portion of the tanning lamp rays emanating from the lower tanning lamps 110 to pass therethruogh.

As illustrated more specifically in FIG. 9, the lower unit 12 typically further includes a substantially transparent lower supporting platform 114 extending substantially across the lower chamber tanning aperture for supporting the intended user while allowing at least part of the tanning light rays emanating from the lower tanning lamps 110 to pass therethrough. The lower supporting platform 114 is movably coupled to the lower lamp chamber 112 for movement relative thereto about between a lower platform opened configuration shown in FIG. 9 allowing access to the lower chamber inner volume and a lower platform closed configuration preventing access to the lower chamber inner volume.

Typically, the lower supporting platform 114 is pivotally attached to the lower lamp chamber 112 for pivotal movement relative thereto about a lower platform pivotal axis 116. Typically, a lower platform supporting means is coupled to the lower platform 114 for selectively maintaining the lower supporting platform 114 in the lower platform opened configuration shown in FIG. 9.

The lower platform supporting means may take any suitable form including that of at least one lower platform piston-type cylinder 118 attached at a longitudinal end thereof respectively to the lower supporting platform 114 and the lower lamp chamber 112. Typically, a lower platform releasable locking means is coupled to the lower supporting platform 114 for releasably locking the lower supporting platform 114 in the lower platform closed configuration.

As illustrated more specifically in FIG. 11, the upper unit 16 typically includes at least one and preferably a plurality of upper tanning lamps 120 positioned in a substantially parallel side-by-side relationship relative to each other. The upper unit 16 also includes an upper lamp chamber 122 defining an upper chamber inner volume for housing the upper tanning lamps 120. The upper lamp chamber has an upper chamber tanning aperture 124 extending therethrough for allowing at least part of the tanning light rays emanating from the upper tanning lamps 120 to pass therethrough.

The upper unit 16 also includes an upper platform 126 movably coupled to the upper lamp chamber 122 for movement relative thereto between an upper platform opened configuration shown in FIG. 11 allowing access to the upper chamber inner volume and an upper platform closed configuration preventing access to the upper chamber inner volume.

Typically, the upper platform 126 is pivotally attached to the upper lamp chamber 122 for pivotal movement relative thereto about an upper platform pivotal axis 127. Also, the upper unit 16 is typically further provided with an upper platform pivotal damping means coupled to the upper platform 126 for damping the pivotal movement of the upper platform 126 toward the upper platform opened configuration. The upper platform damping means may take any suitable form including an upper platform piston-type cylinder 128 attached at longitudinal ends thereof respectively to the upper platform 126 and the upper lamp chamber 122.

The upper and lower platform releasable locking means may take any suitable form. FIGS. 12 through 16 illustrate a possible embodiment for the upper and lower platform releasable locking means. Each platform releasable locking means typically includes a locking catch or tongue 130 pivotally mounted for pivotal movement about a tongue axis 132 between a locked configuration wherein it releasably retains a locking pin 134 and an unlocked configuration wherein it releases the locking pin 134. A tongue biasing means such as a helicoidal-type spring 136 is coupled to the locking tongue 130 for biasing the latter towards the locked configuration shown in FIG. 14.

A tongue actuating mechanism is provided for allowing an intended user to selectively move the locking tongue 130 towards the tongue unlocked configuration. The tongue actuating means typically includes a lock component 138 having a mechanism allowing selective rotation thereof upon insertion therein of a suitable key 140.

The lock component 138 is mechanically coupled to a pivoting component 142. An actuating cable 144 is attached at each end thereof respectively to the pivoting component 142 and to the locking tongue 130 through pulleys 146 for converting the pivotal movement of the pivot component 142 and hence of the lock component 138 into pivotal movement of the locking tongue 130.

Referring now more specifically to FIGS. 17 through 19, there is shown a tanning system 10′ in accordance with an alternative embodiment of the invention. The tanning system 10′ is substantially similar to the tanning system 10 and, hence, similar reference numerals will be used to denote similar components.

The tanning system 10′ includes at least one and preferably a set of high pressure lamps 148 positioned within the upper lamp chamber 122 substantially adjacent the upper side end 28. A corresponding set of ultra-violet filters 150 are mounted to the upper platform 126. The ultra-violet filters 150 are configured, positioned and sized so as to filter the light rays emanating from the high pressure lamps 148 when the upper platform 126 is in the upper platform closed configuration.

Preferably, a switch means is provided for sensing the present of the ultra-violet filters 150 and only allowing the high pressure lamps 148 to be turned on upon the detection of the presence of the ultra-violet filters 150. The switch means may take any suitable form including filter sensors 152 electrically coupled to a suitable electronic or electric circuitry.

1. A counterbalancing device for use in conjunction with a tanning system, said tanning system generating tanning light rays for tanning an intended user, said tanning system having a stationary lower unit and a pivotable upper unit pivotally attached to said lower unit by a unit hinge for pivotable movement therebetween about a unit pivot axis, said upper and lower units respectively defining longitudinally extending upper and lower unit front and rear ends, said upper and lower units respectively defining transversally extending upper and lower unit opposed side ends, said counterbalancing device comprising:
a first component, said first component having a first component base wall, said first component base wall defining a first component inner surface and a substantially opposed first component outer surface;
a second component, said second component having a second component base wall, said second component base wall defining a second component inner surface and a substantially opposed second component outer surface;
a device hinge means for hingedly connecting said first and second components together for relative pivotal movement therebetween about a component pivot axis between a device closed configuration wherein said first and second component inner surfaces are in a substantially parallel and proximal relationship relative to each other and a device open configuration wherein said first and second component inner surfaces are in a substantially angled and spaced relationship relative to each other;
a biasing means for generating a biasing force urging said first and second components towards said device open configuration, said biasing means having a biasing means first abutment segment, a substantially opposed biasing means second abutment segment and a biasing means biasing segment extending integrally therebetween;
a spring-to-component coupling means for operatively coupling said spring first and second abutment segments in abutting contact respectively with said first and second component inner surfaces;
a component-to-unit coupling means for operatively coupling said first and second components respectively to said upper and lower units;
whereby said biasing means is calibrated so that said biasing force substantially counterbalances the weight of said upper unit.

2. A counterbalancing device as recited in claim 1 wherein said biasing means includes a torsion spring, said torsion spring having a spring first abutment segment, a substantially opposed spring second abutment segment and a spring coiled segment extending integrally therebetween, said spring first and second abutment segments being configured, sized and positioned for abuttingly contacting respectively said first and second component inner surfaces, said spring coiled segment being able to generate a biasing force for biasing said first and second components towards said device open configuration.

3. A counterbalancing device as recited in claim 2 wherein said biasing means includes at least two independent torsion springs operatively coupled to said first and second components so as to independently work in parallel to commonly generate said biasing force.

4. A counterbalancing device as recited in claim 2 wherein said spring-to-component coupling means includes
a first “U”-shaped bolt and a second “U”-shaped bolt each having a pair of bolt legs;
a pair of first spring attachment apertures extending through said first component base wall, said first spring attachment apertures being configured, sized and positioned for receiving said pair of bolt legs of said first “U”-shaped bolt;
a pair of second spring attachment apertures extending through said second component base wall, said second spring attachment apertures being configured, sized and positioned for receiving said pair of bolt legs of said second “U”-shaped bolt.

5. A counterbalancing device as recited in claim 1 wherein said first component base wall defines a pair of longitudinally opposed first base wall end edges and said second component base wall defines a pair of longitudinally opposed second base wall end edges; said first component base wall being provided with a first base wall end flange extending substantially perpendicularly from each of said first base wall end edges; said second component base wall being provided with a second base wall end flange extending substantially perpendicularly from each of said second base wall end edges; each of said first base wall end flanges being provided with a first end flange hinge segment projecting substantially outwardly therefrom; each of said second base wall end flanges being provided with a second end flange hinge segment projecting substantially outwardly therefrom; each pair of adjacent first and second flange hinge segments being substantially in register with each other in a substantially proximal and parallel relationship relative to each other; said device hinge means including a device hinge axle pivotally connecting together each pair of adjacent first and second flange hinge segments.

6. A counterbalancing device as recited in claim 1 wherein said unit hinge includes
an upper unit hinge arm extending from said upper unit rear end substantially adjacent each of said upper unit side ends;
a lower unit hinge arm extending from said lower unit rear end substantially adjacent each of said lower unit side ends;
a unit hinge axle pivotally connecting together each pair of adjacent upper and lower unit hinge arms.

7. A counterbalancing device as recited in claim 6 wherein said first component base wall defines a pair of longitudinally opposed first base wall end edges and said second component base wall defines a pair of longitudinally opposed second base wall end edges; said first component base wall being provided with a first base wall end flange extending substantially perpendicularly from each of said first base wall end edges; said second component base wall being provided with a second base wall end flange extending substantially perpendicularly from each of said first base wall end edges; each of said first base wall end flange being provided with a first end flange hinge segment projecting substantially outwardly therefrom; each of said second base wall end flange being provided with a second end flange hinge segment projecting substantially outwardly therefrom; each pair of adjacent first and second flange hinge segments being substantially in register with each other in a substantially proximal and parallel relationship relative to each other; said device hinge means including a device hinge axle pivotally connecting together each pair of adjacent first and second flange hinge segments; said device and unit hinge axles being in a substantially collinear relationship relative to each other.

8. A counterbalancing device as recited in claim 7 wherein
said first and second end flange hinge segments are respectively provided with first and second end flange hinge apertures extending therethrough;
said upper and lower unit hinge arms are respectively provided with upper and lower unit hinge apertures extending therethrough;
a pair of adjacent first and second end flange hinge apertures is positioned substantially in register with a corresponding pair of upper and lower unit hinge apertures so that an hinge bolt acts as both said device and unit hinge axles.

9. A counterbalancing device as recited in claim 1 further comprising
an upper unit hinge arm extending from said upper unit rear end substantially adjacent each of said upper unit side ends; each of said upper unit hinge arm including an upper arm first segment attached to said upper unit, an upper arm second segment extending substantially downwardly at an angle relative to said upper arm first segment from a positioned located substantially adjacent said upper unit rear end, an upper arm third segment extending substantially rearwardly from said upper arm second segment;
a lower unit hinge arm extending from said lower unit rear end substantially adjacent each of said lower unit side ends; each of said lower unit hinge arm including an lower arm first segment attached to said lower unit, an lower arm second segment extending substantially upwardly at an angle relative to said lower arm first segment from a positioned located substantially adjacent said lower unit rear end, an lower arm third segment extending substantially rearwardly from said lower arm second segment;
a unit hinge axle pivotally connecting together said upper and lower arm second segments so that said upper and lower arm first and third segments pivot together in a scissor-like manner.

10. A counterbalancing device as recited in claim 9 further comprising a third segment-to-component coupling means extending between said upper and lower arm third segments and said second and first components for coupling said upper and lower arm third segments respectively to said second and first components.

11. A counterbalancing device as recited in claim 9 wherein said first component base wall defines a pair of longitudinally opposed first base wall end edges and said second component base wall defines a pair of longitudinally opposed second base wall end edges; said first component base wall being provided with a first base wall end flange extending substantially perpendicularly from each of said first base wall end edges; said second component base wall being provided with a second base wall end flange extending substantially perpendicularly from each of said first base wall end edges; each of said first base wall end flange being provided with a first end flange hinge segment projecting substantially outwardly therefrom; each of said second base wall end flange being provided with a second end flange hinge segment projecting substantially outwardly therefrom; each pair of adjacent first and second flange hinge segments being substantially in register with each other in a substantially proximal and parallel relationship relative to each other; said device hinge means including a device hinge axle pivotally connecting together each pair of adjacent first and second flange hinge segments; said device and unit hinge axles being in a substantially collinear relationship relative to each other; said counterbalancing device also including a third segment-to-component coupling means extending between one of said upper and lower arm third segments and said second and first components for coupling said one of said upper and lower arm third segments respectively to said second and first components.

12. A counterbalancing device as recited in claim 11 wherein said third segment-to-component coupling means includes
an upper arm coupling aperture and a lower arm coupling aperture extending respectively through said one of said upper and lower arm third segments;
a first end flange coupling aperture and a second end flange coupling aperture extending respectively through said first and second end flanges;
a first arm-to-flange coupling bolt and a second arm-to-flange coupling bolt extending respectively though said upper arm coupling aperture and said second end flange coupling aperture and through said lower arm coupling aperture and said first end flange coupling aperture;
a first end flange hinge aperture and a second end flange hinge aperture extending respectively through said first and second end flanges;
an upper unit hinge aperture and a lower unit hinge aperture extending respectively through said upper and lower unit hinge arms; said pair of adjacent first and second hinge apertures is positioned substantially in register with a corresponding pair of upper and lower unit hinge apertures so that an hinge bolt acts as both said device and unit hinge axles.

13. A counterbalancing device as recited in claim 12 further comprising a biasing force adjustment means for allowing adjustment of the magnitude of said biasing force.

14. A counterbalancing device as recited in claim 12 wherein either one of said upper or lower arm coupling apertures has a substantially elongated and vertical configuration.

15. A counterbalancing device as recited in claim 1 wherein said lower unit includes
at least one lower tanning lamp;
a lower lamp chamber defining a lower chamber inner volume for housing said at least one lower tanning lamp, said lower lamp chamber having a lower chamber tanning aperture extending therethrough for allowing at least part of said tanning light rays emanating from said lower tanning lamp to pass therethrough;
a substantially transparent lower supporting platform extending substantially across said lower chamber tanning aperture for supporting said intended user while allowing said at least part of said tanning light rays emanating from said lower tanning lamp to pass therethrough;
said lower supporting platform being movably coupled to said lower lamp chamber for movement relative thereto between a lower platform open configuration allowing access to said lower chamber inner volume and a lower platform closed configuration preventing access to said lower chamber inner volume.

16. A counterbalancing device as recited in claim 15 wherein said lower supporting platform is pivotally attached to said lower lamp chamber for pivotal movement relative thereto about a lower platform pivotal axis

17. A counterbalancing device as recited in claim 15 further comprising a lower platform supporting means coupled to said lower supporting platform for selectively maintaining said lower supporting platform in said lower platform open configuration.

18. A counterbalancing device as recited in claim 15 further comprising a lower platform releasable locking means coupled to said lower supporting platform for releasably locking said lower supporting platform in said lower platform closed configuration.

19. A counterbalancing device as recited in claim 1 wherein said upper unit includes
at least one upper tanning lamp;
a upper lamp chamber defining a upper chamber inner volume for housing said at least one upper tanning lamp, said upper lamp chamber having a upper chamber tanning aperture extending therethrough for allowing at least part of said tanning light rays emanating from said upper tanning lamp to pass therethrough;
a substantially transparent upper platform extending substantially across said upper chamber tanning aperture for allowing said at least part of said tanning light rays emanating from said upper tanning lamp to pass therethrough;
said upper platform being movably coupled to said upper lamp chamber for movement relative thereto between a upper platform open configuration allowing access to said upper chamber inner volume and a upper platform closed configuration preventing access to said upper chamber inner volume.

20. A counterbalancing device as recited in claim 19 wherein said upper platform is pivotally attached to said upper lamp chamber for pivotal movement relative thereto about an upper platform pivotal axis

21. A counterbalancing device as recited in claim 19 further comprising a upper platform pivotal damping means coupled to said upper platform for damping the pivotal movement of said upper platform towards said upper platform open configuration.

22. A counterbalancing device as recited in claim 19 further comprising a upper platform releasable locking means coupled to said upper platform for releasably locking said upper platform in said upper platform closed configuration.

23. A counterbalancing device as recited in claim 19 further comprising
a high pressure lamp positioned within said upper lamp chamber substantially adjacent one of said upper unit side ends;
a UV filter mounted to said upper platform, said UV filter being configured, positioned and sized so as to filter light rays emanating from said high pressure lamp when said upper platform is in said upper platform closed configuration.

24. A counterbalancing device as recited in claim 23 further comprising a switch means for sensing the presence of said UV filter and only allowing said high pressure lamp to be turned on upon the detection of the presence of said UV filter.
